(19) United States
(12) Patent Application Publication
(10) Pub. No.: US 2026/0346036 A1
(43) Pub. Date: Nov. 5, 2026

(54) APPLICATOR DEVICE FOR APPLYING MAGNETIC FIELDS FOR MAGNETIC FIELD THERAPY

(71) Applicant: gbo Medizintechnik AG, Rimbach (DE)

(72) Inventor: Michael KESSLER, Wald-Michelbach (DE)

(21) Appl. No.: 19/488,399

(22) PCT Filed: May 6, 2024
(86) PCT No.: PCT/EP2024/062474

§ 371 Date: Nov. 26, 2025

(30) Foreign Application Priority Data
May 30, 2023 (EP) 23176055.4

(51) Int. Cl. A61N 2/00; A61F 9/02; A61N 2/02

(52) U.S. Cl. CPC A61N 2/006; A61F 9/029; A61N 2/02

(57) ABSTRACT

The invention relates to an applicator device for applying magnetic fields for magnetic field therapy, said device comprising a plurality of applicators for applying magnetic fields. The applicator device has an elastically deformable headband comprising a first arm, a second arm, and a connecting portion that connects the first arm to the second arm, wherein the headband is designed to be worn in the head region of a person when the applicator device is in use, such that the connecting portion extends over the face of the person, in particular over the eyes of the person, and the arms extend towards the ears, wherein a pair of eye applicators are arranged in or on the connecting portion, and an ear applicator is arranged in or on each of the arms, wherein each applicator has a coil for generating a magnetic field when current flows through the coil.

### DESCRIPTION

The invention relates to an applicator device for applying magnetic fields for magnetic field therapy having multiple applicators for applying magnetic fields.

Magnetic field therapies, in which an organism, especially a human organism, is ex-posed to a temporally varying magnetic field, are presently being used in many sec-tors to promote well-being and health. The magnetic fields, which are usually low-frequency, are usually generated via current-carrying coils. For example, a device for emitting electromagnetic fields is described in DE 10 2018 119 019A1, wherein this device has two actuators for emitting electromagnetic fields, wherein these two actuators are each configured to emit electromagnetic fields of different frequencies. Devices are known from the prior art which are used to expose the eye area of a person to be treated to a magnetic field. For example, a device for generating an electromagnetic field having a frequency in the range of the vibrational frequency of the earth's magnetic field is described in WO 2010/022419A1, wherein an electrical line for building up the electromagnetic field is arranged in a pair of glasses, so that in the usage position of the glasses, the electromagnetic field is generated in the eye area of the person to be treated. US 2019/0299020A1, WO 2020/206032A1, and WO 2019/173866 A1 describe further prior art.

In addition to applicators that are used to treat the eyes of a person using a magnetic field, applicators are also known that are used to treat the ears of a person using electromagnetic fields.

The simultaneous treatment of the ears of a person and the eyes of a person using magnetic fields is usually not possible. Magnetic field therapy devices are known which have a separate signal generating unit, wherein different applicators can be connected to the signal generating unit, wherein the signal generating unit is used to generate the current signal for the current-carrying coils of the applicator connected to the device. However, in such magnetic field therapy devices only one of the two applicators, namely either the eye applicator or the ear applicator, can be connected to the signal generating unit. In addition, the applicators are often not suitable to be used jointly in terms of their design or anatomy. This is true in particular because applicators for the eyes are often designed in the form of glasses, the temples of which are placed on and behind the ears, so that the additional arrangement of a magnetic field therapy device or applicator for the ears designed like headphones is often uncomfortable for the person to be treated when used in com-bination with the eye applicator.

There is therefore a need for an applicator device that is suitable for the treatment of the eyes and for the treatment of the ears and in particular for the combined treatment of eyes and ears.

This object is achieved by an applicator device which has the features of claim 1. Advantageous refinements are the subject matter of the dependent claims.

The applicator device according to the invention is used for treating the eyes of a person and for treating the ears of a person and in particular for combined treatment of the eyes and ears of a person. For this purpose, the applicator device has multiple applicators for applying magnetic fields, in particular for applying temporally varying magnetic fields, in particular for applying electromagnetic fields. The applicator device has an elastically deformable support bracket having a first leg, a second leg, and a connecting section connecting the first leg to the second leg, wherein the support bracket is adapted to be worn in the head area of a person during use of the applicator device, thus in the usage position, such that the connecting section extends over the face of the person, in particular the eyes of the person, and the legs extend in the direction of the ears, wherein a pair of eye applicators is arranged in or on the connecting section and an ear applicator is arranged in or on each of the legs, wherein the respective applicator has a coil for generating a magnetic field when a current flows through the coil. The pair of eye applicators is thus used to treat the eyes of the person and the ear applicators are used to treat the ears of the person.

This design of the applicator device allows a high level of wearing comfort for the person to be treated and the treatment of the eyes, the ears, or the simultaneous treatment of the eyes and ears of the person. Accordingly, it is no longer necessary to provide two different magnetic field therapy devices or separate applicators, namely a magnetic field therapy device or an applicator for the eyes and a further magnetic field therapy device or a further applicator for the ears, but only a single applicator device, namely the combined applicator device according to the invention for these uses. Because the support bracket is elastically deformable, the support bracket fits itself optimally to the dimensions of the head of the person to be treated and ensures a good hold of the applicator device even without further holding means, since the restoring forces of the elastically deformable support bracket ensure that the legs have a good hold on the head of the person to be treated. Due to the design having the elastically deformable support bracket, it is therefore not necessary to provide further fastening or holding means on the applicator device.

A coil as defined in the present invention comprises at least one conductor having at least one winding or turn. For example, the respective coil can be a flat coil, a worm coil, an air coil, or a ring coil. Preferably, the respective coil has at least 30 turns. For example, the conductor can be a copper wire. Preferably, the respective coil consists of a single, wound conductor. Preferably, the coils of the ear applicators have a larger number of windings than the coils of the eye applicators.

Preferably, the connecting section is supported in the usage position on the nose of the person to be treated. This also ensures that the eye applicators are in a defined position with respect to the eyes of the person to be treated and the hold of the applicator device is improved.

It is considered to be particularly advantageous if the legs for use in the head area of the person have to be bent apart against a restoring force, by which a particularly good hold in the head area is achieved. In this context, it is considered to be particularly advantageous if the legs in a starting state of the applicator device are directed toward one another, for example bent by more than 190°, and have to be bent apart to use the applicator device.

It is furthermore considered to be advantageous if the support bracket is bent U-shaped, in particular the entire applicator device is substantially U-shaped, thus no further fastening components or supporting components, such as a headband, fastening strap, or rubber band, are provided.

It is considered to be particularly advantageous if the coils of the eye applicators are arranged fixed in the support bracket. It has been shown in practice that the position of the eyes in relation to one another does not vary so much between different persons that it is necessary to adjust the coils with regard to their arrangement in the support bracket. Accordingly, the desired magnetic field treatment in the eye area can also be achieved using stationary coils for different persons. This makes the design of the applicator device much easier, which can also save weight, which has a beneficial effect on the wearing comfort of the applicator device.

It is considered to be advantageous if the first ear applicator is arranged in the area of a free end of the first leg and the second ear applicator is arranged in the area of a free end of the second leg.

In a preferred refinement it is provided that the respective ear applicator is mounted in the respective leg to be displaceable along the respective leg. It has been shown in practice that by adjusting the two ear applicators along the respective leg, a very good fitting of the applicator device to the anatomy of the head of the respective person can be achieved. It has been shown that a displaceability along the respective leg is sufficient to achieve a sufficient fitting of the applicator device or the arrangement of the applicators. For example, it has been shown that it is not necessary to adjust the ear applicators in other spatial directions in relation to the eye applicators. Accordingly, it is considered to be particularly advantageous if the respective ear applicator is only displaceable along the respective leg, by which in turn a particularly simple design of the applicator device can be achieved and also weight can be saved, which in turn has an advantageous effect on the wearing comfort.

It is considered to be particularly advantageous if the legs are integrally formed with the connecting section. Accordingly, the legs are preferably connected without hinges to the connecting section.

Preferably, the support bracket consists of an elastically deformable material, in particular of a plastic, preferably of acrylonitrile-butadiene-styrene (ABS).

It is considered to be particularly advantageous if a receptacle area for the coils of the eye applicators is provided in the support bracket, as well as a channel for arranging the necessary electrical wiring, wherein the electrical wiring is guided in each case up to the legs of the support bracket and from there to the ear applicators.

Preferably, the support bracket forms a housing shell having a receptacle space for accommodating the coils of the eye applicators and/or a receptacle space for the wiring, wherein the housing shell is closed using a corresponding housing cover, by which the wiring and/or the coils of the eye applicators are protected from external influences. The housing cover is preferably clipped onto the housing shell. In this respect, it is considered to be particularly advantageous if the lid is also bent U-shaped. The lid is preferably also made of an elastic material, in particular ABS.

In an advantageous refinement it is provided that the respective ear applicator comprises a housing, wherein the coil of the respective ear applicator is accommodated in the housing. In the area of the ear applicator, further components of the applicator device can also be arranged or attached. For example, it is considered to be particularly advantageous if an electrical connection to an external power supply, in particular in the form of a cable, is attached in the area of one or both ear applicators. This is because the wiring in this area is least disturbing for the person to be treated and also tensile forces or weight forces in the area of the ear applicators are more advantageous in terms of wearing comfort than would be the case if the wiring were attached in the area of the connecting section.

To increase the wearing comfort of the applicator device, it is considered to be particularly advantageous if the connecting section has a structure fitted to the shape of the nose in its area resting on the nose, for example, in the form of a recess. However, it is also conceivable that a separate nose pad or a nose bridge is attached to the connecting section, as is known, for example, from conventional glasses. Preferably, the connecting section has a recess in the area coming into contact with the nose.

It is considered to be particularly advantageous if the connecting section has a viewing opening for the respective eye. When using the applicator device, the person can therefore still observe the environment, read, or perform other activities with the eyes. It is considered particularly advantageous in this context if the coils of the eye applicators are formed as hollow coils or ring coils, so that these coils can be arranged circumferentially around the viewing opening. This allows an optimum application of the magnetic field in the area of the respective eye and the person can still look through the hollow core of the coil and the provided viewing opening.

Against the background of the closest possible arrangement of the coils of the eye applicators to the eyes of the person to be treated, it is considered to be particularly advantageous if the connecting section has two receptacle pockets, wherein the coil of the respective eye applicator is received in the receptacle pockets, wherein the respective receptacle pocket is formed as a bulge protruding in the direction of a rear side of the connecting section, which is to be faced toward the person. The rear side of the receptacle pocket is therefore closed and the coil is thus covered.

In this case, it is considered to be particularly advantageous if the connecting section has a hollow cylindrical bearing section associated with the respective eye applicator, wherein the coil of the respective eye applicator is pushed onto or wound around the corresponding bearing section for radial mounting of the coil. Preferably, the respective bearing section is formed by a ring-shaped projection protruding in the direction of the front side. The hollow cylindrical bearing section preferably has an oval cross section.

It is considered to be particularly advantageous if a trim element covering the connecting section is attached to the connecting section on a front side to be faced away from the person, wherein the trim element is attached to the connecting section and the legs are elastically deformable independently of the trim element, in particular in such a way that it is possible to bend the legs apart up to a maximum width, whereby the trim element prevents further bending beyond the maximum width. This design has the advantage that the trim element can be formed largely independently of the support bracket, which is elastically deformable, without impairing the functionality of the elastically deformable support bracket with regard to optimum shaping to the head area of the person. Accordingly, the trim element can be designed largely under design aspects and in particular also as dimensionally stable or rigid, without taking into consideration functional aspects with regard to the shaping to the head of the person, since the functional aspects are provided by the elastically deformable support bracket. In that the trim element is fastened, preferably exclusively, to the connecting section, a functionality of the elastically deformable legs is not impaired by the trim element. Accordingly, the trim element in a preferred embodiment can be more dimensionally stable than the support bracket, for example, it can consist of a stiffer material. The trim element can form the lid for the housing shell.

Preferably, in a central area of the connecting section, the trim element is fastened thereon, for example, screwed thereon. Preferably, the screw connection is from the rear side so that the screw connection is not visible from the front side.

Furthermore, it is considered to be particularly advantageous if the trim element is transparent at least in the area of the viewing openings. Preferably, the trim element is made of a transparent material.

The material of the trim element can be, for example, a glass, in particular a plastic glass, such as acrylic glass. Preferably, the material of the trim element is transparent ABS.

In addition, there is also the possibility that the trim element in the area of the viewing openings is provided with a certain glasses prescription to compensate for a di-opter compensation in case of a visual impairment of the person to be treated. This makes it possible for the person wearing the applicator device to read, watch TV, or perform other activities with the eye during treatment despite a visual impairment. By stocking different trim elements, a trim element matching the visual impairment of the person to be treated can be selected and attached to the connecting section. Optimum adjustment is usually not necessary, as is also known from reading glasses, for example.

It is entirely conceivable that the trim element is detachably attached to the connecting section. This enables easy replacement of the trim element. Thus, a simple adjustment of the design of the applicator device and/or, in the case of suitable trim elements, an adjustment to the visual impairment or defective vision of the person can be made.

It is considered to be particularly advantageous if the trim element is mirrored on the outside to be faced away from the person.

Since different magnetic fields are usually used for the treatment of the eyes and for the treatment of the ears during magnetic field therapy, i.e. magnetic fields are used which differ, for example, in terms of their frequency and/or their strength and/or their pulse width and/or their pulse frequency, it is considered to be particularly advantageous if the applicator device is configured to actuate the ear applicators and the eye applicators differently, i.e. to design the current signal flowing through the respective coil differently. Accordingly, it is considered to be particularly advantageous if the applicator device for the ear applicators and the eye applicators are each actuatable via separate signal generating units or have separate connections. Accordingly, the coils of the eye applicators are isolated from the coils of the ear applicators with respect to the current signal. On the other hand, the coils of the ear applicators are preferably connected to one another with respect to the current signal, so that the same magnetic field is generated by the respective coil. The same applies to the coils of the eye applicators.

Preferably, the signal generating units for the applicators are integrated in the applicator device, so that a separate, for example stationary, signal generating unit can be dispensed with. The signal generating unit is therefore worn together with the signal generating unit on the head. Only one power source is then required, preferably a DC source, which is electrically connected to the respective signal generating unit. The power source can be a separate device connected to the applicator device by a connecting cable. However, in principle, batteries or accumulators, for example in the form of a lithium-ion battery, are also conceivable as a power source. This enables the applicator device to be used as autonomously as possible.

It is considered to be particularly advantageous if the applicator device has a first signal generating unit for the respective eye applicator or a common first signal generating unit for both eye applicators, wherein the first signal generating units or the common first signal generating unit are electrically connected to the coils of the eye applicators, wherein the first signal generating units or the common first signal generating unit are configured to generate the current signal flowing through the coils of the eye applicators, wherein the first signal generating units or the common first signal generating unit are integrated in one of the ear applicators. This in turn has the advantage that weight is saved in the area of the connecting sections and yet the corresponding signal generating unit is directly integrated in the applicator device, so that an external signal generating unit can be dispensed with. This enables the applicator device to be used as autonomously as possible. Thus, it is entirely conceivable that the applicator device only has to be connected to a power source, since the signal formation or signal generation for the current-carrying coils takes place in the applicator device. This also opens up the possibility of actuating the applicator device as simply as possible in order to generate the desired signal form, wherein this actuation can be carried out, for example, using a mobile device, in particular a cell phone. Against this background, it is also considered to be particularly advantageous if the applicator device has a communication interface that allows a communication connection to a mobile terminal. This communication interface is preferably designed as a wireless communication interface.

The respective first signal generating unit may also comprise a storage device in which preset therapy programs and corresponding current signal forms are stored.

With regard to the first signal generating units or the common first signal generating unit, it is considered to be particularly advantageous if this signal generating unit is formed within a housing of the respective ear applicator.

It is considered to be particularly advantageous if the applicator device has a second signal generating unit for the respective ear applicator or a common second signal generating unit for both ear applicators, wherein the second signal generating units or the common second signal generating unit are electrically connected to the coils of the ear applicators, wherein the second signal generating units or the common second signal generating unit are configured to generate the current signal flowing through the coils of the ear applicators, wherein the second signal generating units or the common second signal generating unit are integrated in one of the ear applicators.

The advantages and advantageous refinements mentioned for the first signal generating unit apply correspondingly to the second signal generating unit.

It is considered to be particularly advantageous if at least one of the signal generating units is integrated in the respective ear applicator, in particular the common first signal generating unit is integrated in the one ear applicator and the common second signal generating unit is integrated in the other ear applicator. Thus, the different signal generating units are spatially separated from one another, by which a possible interaction between the signal generating units, which could possibly have a negative effect on the corresponding current signal, is avoided or at least attenuated.

It is considered to be particularly advantageous if the respective signal generating unit comprises a printed circuit board having electronic components attached thereon. As a result, the respective signal generating unit can be installed particularly easily in the respective ear applicator, in particular in the respective housing.

It is considered to be particularly advantageous if the coil of the respective ear applicator is soldered directly to the signal generating unit formed in the corresponding ear applicator.

The applicator device is in particular configured to generate a magnetic field in the area of the respective applicator, wherein an effective value of this magnetic field is between 40 μT and 1 mT.

Preferably, the applicator device is configured to generate a magnetic field having a pulse frequency of less than or equal to 500 Hz.

In the following figures, the invention is explained in more detail on the basis of exemplary embodiments, without being restricted thereto. In the figures:

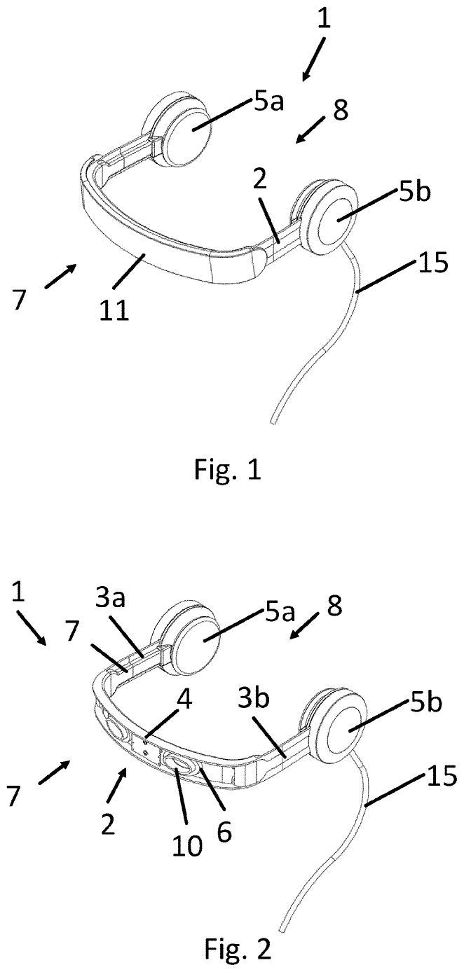

FIG. 1 shows a first embodiment of an applicator device for treating the eyes and ears of a person in a view diagonally from the front,

FIG. 2 shows the applicator device according to FIG. 1 without front-side trim element,

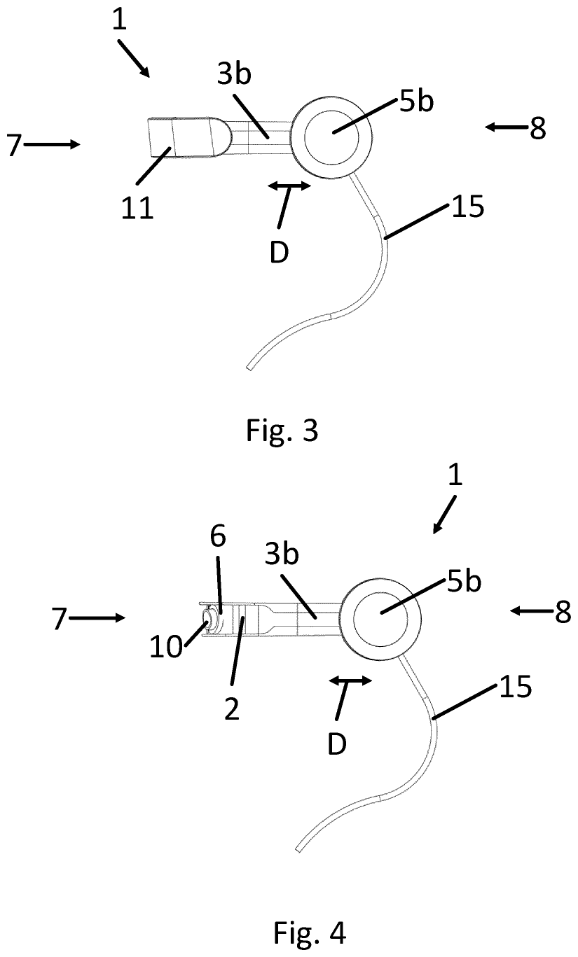

FIG. 3 shows the applicator device according to FIG. 1 in a side view,

FIG. 4 shows the applicator device according to FIG. 1 in a side view without front-side trim element,

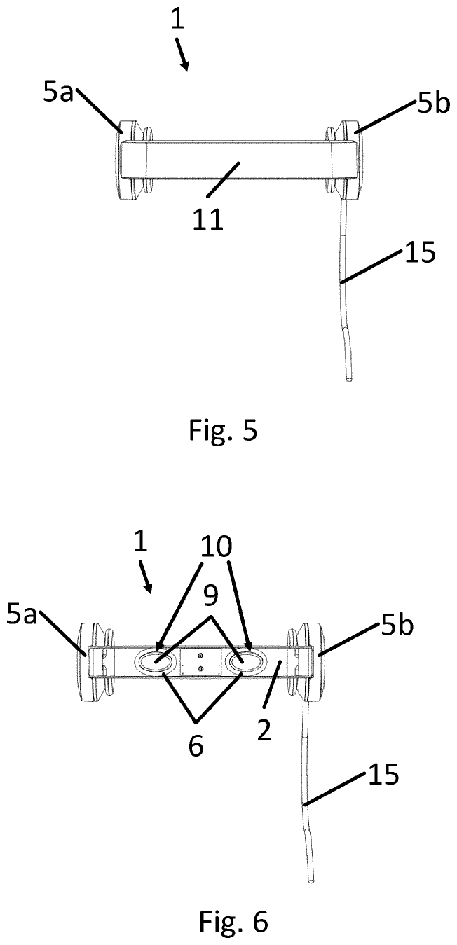

FIG. 5 shows the applicator device according to FIG. 1 in a front view,

FIG. 6 shows the applicator device according to FIG. 1 in a front view without front-side trim element,

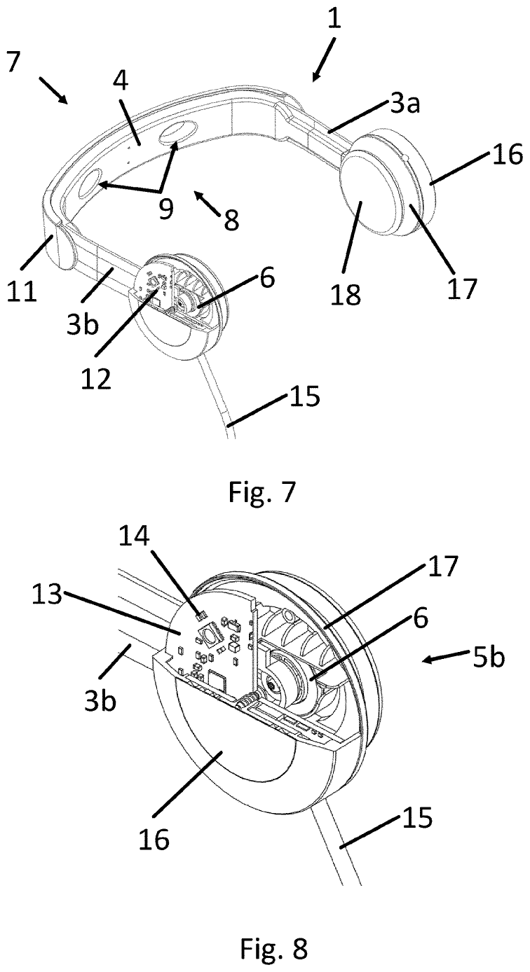

FIG. 7 shows the applicator device according to FIG. 1 in a representation in partial section in a view diagonally from the rear,

FIG. 8 shows a detail of FIG. 7 in an enlarged representation,

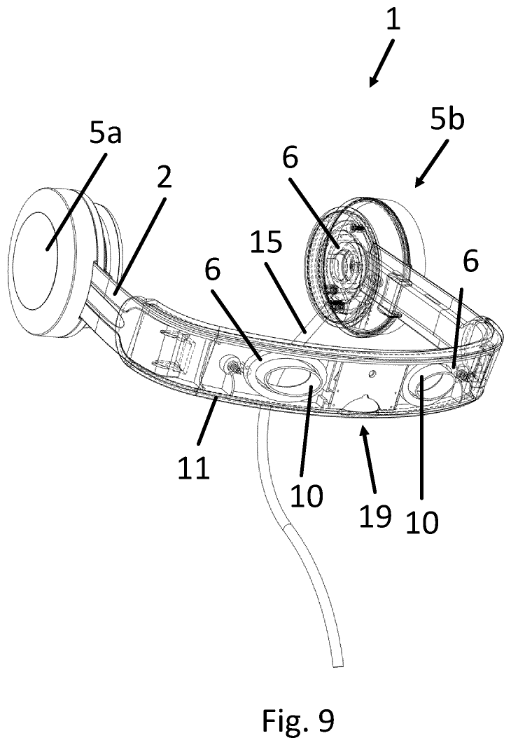

FIG. 9 shows a second embodiment of the applicator device in a view diagonally from the front.

FIGS. 1 to 8 show a first embodiment of an applicator device 1, wherein this applicator device 1 is designed to apply a magnetic field both in the area of the eyes of a person to be treated and in the area of the ears of a person to be treated. The applicator device 1 is designed to be worn in the head area of a person to be treated. For this purpose, the applicator device 1 has an elastically deformable support bracket 2 having a first leg 3a, a second leg 3b, and a connecting section 4 connecting the first leg 3a to the second leg 3b. The support bracket 2 as such is U-shaped and adapted to be worn in the head area of a person when using the applicator device 1, in this sense in the usage position, in such a way that the connecting section 4 is supported on the nose of the person and the legs 3a, 3b extend in the direction of the ears. Multiple applicators are mounted in the support bracket 2, wherein a pair of eye applicators are mounted in the connecting section 4 and an ear applicator 5a, 5b is mounted on each of the legs 3a, 3b. The respective applicator comprises a coil 6 for generating a magnetic field when a current flows through the coil 6, wherein the pair of eye applicators is used for treating the eyes of the person and the ear applicators 5a, 5b are used for treating the ears of the person, and the coils 6 are configured for this purpose to apply a magnetic field in the corresponding area. The coils 6 are each a wound conductor in the form of a copper wire, wherein the coils 6 of the eye applicators each have 100 windings and the coils 6 of the ear applicators 5a, 5b each have 60 windings.

The legs 3a, 3b are integrally formed with the connecting section 4, as can be seen in particular from FIG. 2. In the area of the connecting section 4, the coils 6 of the eye applicators are arranged fixed, whereas the first ear applicator 5a and the second ear applicator 5b in the area of a free end of the respective leg 3a, 3b are linearly displaceable along the respective leg 3a, 3b and thus variable in position. This displaceability is indicated by the double arrow D in FIGS. 3 and 4. Due to this design, a simple fitting of the applicator device 1 to the respective anatomy of the head of the person to be treated can be made.

As can be seen in particular in FIGS. 2 and 4, the connecting section 4 has two annular projections protruding in the direction of a front side 7 to be faced away from the person, wherein the respective projection forms a hollow cylindrical bearing section 10, onto which the annular coil 6 of the respective eye applicator is pushed or around which it is wound on the front side. The respective bearing section 10 has neither a bottom nor a lid, which results in a viewing opening 9 for the respective eye. This makes it possible for the person wearing the applicator device 1 to look outward through the applicator device 1.

On the front side 7 of the applicator device 1 facing away from the person, a transparent, front-mirrored trim element 11 in the form of a U-shaped visor is attached.

Since the trim element 11 is transparent, in spite of the fact that this trim element 11 completely covers the connecting section and thus also the viewing openings 9 and partially covers the two legs 3a, 3b, it is nevertheless possible for the person wearing the applicator device 1 to look outward.

The trim element 11 is designed to be more dimensionally stable than the support bracket 2, wherein, due to the fact that the trim element 11 is only fastened in the area of the connecting section 4 to the support bracket 2, the legs 3a, 3b can nevertheless be deformed. In this case, deformation of the legs 3a, 3b outward, thus away from one another, is restricted by the sections of the trim element 11 overlapping with the legs 3a, 3b. In contrast, inward, thus toward one another, a deformation of the legs 3a, 3b is not restricted by the trim element 11, so that only a maximum opening width of the support bracket 2 is restricted by the trim element 11.

The applicator device 1 has a common first signal generating unit 12 for the eye applicators, wherein the common first signal generating unit 12 is electrically connected to the coils 6 of the eye applicators, wherein the common first signal generating unit 12 is configured to generate the current signal flowing through the coils 6 of the eye applicators, wherein the common first signal generating unit 12 is integrated in the left ear applicator 5b. This design is shown in more detail in FIG. 5. The common first signal generating unit 12 is formed by a printed circuit board 13 having electronic components 14 arranged thereon. As can be seen from FIG. 5, a connecting cable 15 is also provided in the area of the left ear applicator 5a, which is used to connect the applicator device 1 to an external power supply, in particular a DC source. The electrical connection of the first signal generating unit 12 to the coils 6 of the eye applicators is carried out by means of a ribbon cable, wherein this ribbon cable is guided in an internal cable channel 7 of the support bracket 2, which extends from the left leg 3b to the connecting section 4. A corresponding cable channel 7 extends from the right leg 3a to the connecting section 4.

In the right ear applicator 5a, a second common signal generating unit is arranged, wherein this second signal generating unit is electrically connected to the coils 6 of the respective ear applicator 5a, 5b, wherein the second signal generating unit is configured to generate the current signal flowing through the coils 6 of the ear applicators 5a, 5b, wherein the left ear applicator 5b is in turn electrically connected via a cable, which is guided in the support bracket 2, to the second common second signal generating unit formed in the first ear applicator 5a. This second signal generating unit in turn comprises a printed circuit board having electronic components attached thereon.

The printed circuit board of the respective signal generating unit 12 is formed circular and thus fitted to the geometric design of a housing of the respective ear applicator 5a, 5b. The respective housing is formed in two parts and has an outer housing shell 16 and an inner housing shell 17. A foam element 18 is attached to the inner housing shell 17, which faces toward the ear of the person to be treated, in order to increase the wearing comfort.

FIG. 9 shows a second embodiment of the applicator device 1, which differs from the first embodiment substantially due to the design of the connecting section 4 and the trim element 11. In the second embodiment, in contrast to the first embodiment, a recess 19 for a nose of the person is provided on both the connecting section 4 and on the trim element 11.

### LIST OF REFERENCE SIGNS

1 applicator device
2 support bracket
3a first leg
3b second leg
4 connecting section
5a first ear applicator
5b second ear applicator
6 coil
7 front side
8 rear side
9 viewing opening
10 bearing section
11 trim element
12 first signal generating unit
13 printed circuit board
14 electronic component
15 connecting cable
16 outer housing shell
17 inner housing shell
18 foam element
19 recess

1-15. (canceled)

16. An applicator device for applying magnetic fields for magnetic field therapy, wherein the applicator device has an elastically deformable support bracket having a first leg, a second leg, and having a connecting section connecting the first leg to the second leg, wherein the support bracket is adapted to be worn in the head area of a person during use of the applicator device such that the connecting section extends over the face of the person, in particular the eyes of the person, and the legs extend in the direction of the ears, wherein a pair of eye applicators is arranged in or on the connecting section and an ear applicator is arranged in or on each of the legs, wherein the respective applicator has a coil for generating a magnetic field when a current flows through the coil.

17. The applicator device as claimed in claim 16, wherein the support bracket is bent U-shaped, wherein the coils of the eye applicators are arranged fixed in the area of the connecting section, and the first ear applicator is arranged in the area of a free end of the first leg and the second ear applicator is arranged in the area of a free end of the second leg.

18. The applicator device as claimed in claim 16, wherein the respective ear applicator is mounted on the respective leg to be displaceable along the respective leg.

19. The applicator device as claimed in claim 16, wherein the legs are integrally formed with the connecting section.

20. The applicator device as claimed in claim 16, wherein the respective ear applicator comprises a housing, wherein the coil of the respective ear applicator is accommodated in the housing.

21. The applicator device as claimed in claim 16, wherein the connecting section has a viewing opening for the respective eye, preferably the coil associated with the respective eye circumferentially surrounds the viewing opening associated with the respective eye.

22. The applicator device as claimed in claim 16, wherein the connecting section has a hollow cylindrical bearing section associated with the respective eye applicator, wherein the coil of the respective eye applicator is pushed onto or wound around the bearing section for radial mounting of the coil.

23. The applicator device as claimed in claim 16, wherein a trim element covering the connecting section is attached to the connecting section on a front side to be faced away from the person, wherein the trim element is attached to the connecting section such that the legs are elastically deformable independently of the trim element.

24. The applicator device as claimed in claim 23, wherein the trim element is detachably attached to the connecting section.

25. The applicator device as claimed in claim 16, wherein the trim element is more dimensionally stable than the support bracket.

26. The applicator device as claimed in claim 16, wherein the trim element is transparent at least in the area of the viewing openings.

27. The applicator device as claimed in claim 16, wherein the applicator device has a first signal generating unit for the respective eye applicator or a common first signal generating unit for both eye applicators, wherein the first signal generating units or the common first signal generating unit are electrically connected to the coils of the eye applicators, wherein the first signal generating units or the common first signal generating unit are configured to generate the current signal flowing through the coils of the eye applicators, wherein the first signal generating units or the common first signal generating unit are integrated in one of the ear applicators.

28. The applicator device as claimed in claim 16, wherein the applicator device has a second signal generating unit for the respective ear applicator or a common second signal generating unit for both ear applicators, wherein the second signal generating units or the common second signal generating unit are electrically connected to the coils of the ear applicators, wherein the second signal generating units or the common second signal generating unit are configured to generate current signal flowing through the coils of the ear applicators, wherein the second signal generating units or the common second signal generating unit are integrated in one of the ear applicators.

29. The applicator device as claimed in claim 16, wherein at least one of the signal generating units is integrated in the respective ear applicator, in particular the common first signal generating unit is integrated in the one ear applicator and the common second signal generating unit is integrated in the other ear applicator.

30. The applicator device as claimed in claim 27, wherein the respective signal generating unit comprises a printed circuit board having electronic components attached thereon.
